(19) United States
(12) Patent Application Publication
(10) Pub. No.: US 2022/0161117 A1
(43) Pub. Date: May 26, 2022

(54) FRAMEWORK FOR RECORDING AND ANALYSIS OF MOVEMENT SKILLS

(71) Applicant: RLT IP LTD., Gosport (GB)

(72) Inventors: David M. Jessop, New Alresford (GB); Peter Robins, Woolton Hill (GB); Jonathan M. Dalzell, Lee-On-The-Solent (GB); Daniel A. Frost, Gosport (GB); Richard E. Collins, Newport (GB)

(21) Appl. No.: 17/623,142

(22) PCT Filed: Jun. 28, 2019
(86) PCT No.: PCT/EP2019/067485

§ 371 Date: Dec. 27, 2021

(51) Int. Cl. A63B 69/36

(52) U.S. Cl. CPC A63B 69/3608

(57) ABSTRACT

A method for a processor to analyze motion data includes receiving times series of raw motion data, hereafter the raw data streams, and generating sparse data streams based on the raw data streams. The method also includes performing a raw data action identification to detect the action in a window of time, which includes modifying one or more first thresholds based on the user information and comparing one or more of the raw data streams in the window with one or more modified first thresholds. The method further includes determining a score of the detected action and providing a visualized feedback to the user based on the score.

### BACKGROUND

Wearable technologies are electronic devices incorporated into clothing or worn on the body. They are often used to monitor an athlete's movements to improve performance. Wearable devices may include motion sensors such as accelerometers and gyroscopes. They may also include biofeedback sensors such as heart rate monitors and temperature sensors.

### BRIEF DESCRIPTION OF THE DRAWINGS

In the drawings:

FIG. 1-1 is a block diagram of a motion sensing system in some examples of the present disclosure.

FIG. 1-2 illustrates a skeletal model for generating the avatar in some examples of the present disclosure.

FIG. 1-3 is a motion capture suit of FIG. 1-1 in some examples of the present disclosure.

FIG. 2 is a swimlane diagram demonstrating how a pod of FIG. 1-1 is configured in some examples of the present disclosure.

FIG. 3 is a flowchart of a method illustrating the operations of the pod of FIG. 1-1 in some examples of the present disclosure.

FIG. 4 illustrates a method for implementing a block in the method of FIG. 3, which generates sparse geometric data streams, in some examples of the present disclosure.

FIG. 5 illustrates a method for implementing a block in the method of FIG. 3, which performs raw data action identification (ID), in some examples of the present disclosure.

FIG. 6 illustrates a method for implementing a block in the method of FIG. 3, which performs raw data action ID, in some examples of the present disclosure.

FIG. 7 illustrates a method for implementing a block in the method of FIG. 3, which performs geometric data action ID, in some examples of the present disclosure.

FIGS. 8, 9, 10, 11-1, and 11-2 are block diagrams illustrating methods for phase ID of different action in some examples of the present disclosure.

FIGS. 12 and 13 are block diagrams illustrating examples of a hierarchical data structure of scores in some examples of the present disclosure.

FIG. 14 is a block diagram illustrating an application of the motion system of FIG. 1 to analyze motion data from multiple users in some examples of the present disclosure.

Use of the same reference numbers in different figures indicates similar or identical elements.

### DETAILED DESCRIPTION

In some examples of the present disclosure, a system is provided to capture and analyze motion data from actions performed by a user. An action may be a physical skill, a technique of a skill, a variation of a technique, or a pose. The motion data include those generated by sensors on a motion capture suit worn by a user. The motion data may also include those generated by a sensor on a piece of sports equipment, such as a golf club, a baseball bat, a tennis racket, a ball, or a torque pedal. The motion data may further include those generated by another electronic device, such as a golf launch monitor. For group activities involving multiple players, the system may capture and analyze motion data from multiple users to coordinate their training. For example, the strokes of multiple rowers may be compared to determine if they are synchronized and provide the appropriate feedback (textual, audio, visual, or a combination thereof), and the movements of an offensive player may be compared against the movements of a defensive player to determine if proper techniques are used and provide the appropriate feedback.

In some examples of the present disclosure, the system creates compressed motion data for animating an avatar that provides visual feedback to the user. The system converts time series of raw motion data (“raw data streams”) from the sensors into sparse time series of geometric data (“sparse geometric data streams”) on limb segments in a skeletal model. The accuracy of the sparse geometric data stream can be specified according to time or relative orientation of the limb segments. At a later time, the system uses the sparse geometric data streams to calculate angles between connected limb segments, angles between the limb segments and an external reference coordinate system, and relative and absolute displacements of segment joints between the limb segments.

The system can be updated automatically using motion data captured from the system. For example, the system may use an observed golf swing as the optimum performance for judging future golf swings. The system may also adjust the identification of a given action based on an observed action.

The system implements each activity (possibly involving equipment, instrumented or otherwise) as (1) actions, (2) phases, and (3) metrics.

The system defines one or more method for detecting an action has been performed and may also classify the type of action performed. The system can configure the detection based on user profile and user biometrics.

The system divides the action into phases from which metrics can be produced. The detection of the phases can be configured according to the action, the user profile, and the user biometrics.

The system defines metrics for the action. Metrics may relate to whole body movement (e.g., center of mass or pressure). Metrics may be kinetic variables (e.g., peak force, impulse). Metrics may relate to external equipment or third-party technology (e.g., launch monitor). Metrics may also be derived from relative and absolute linear and angular displacements, velocities, accelerations, and durations of individual or combined body segments.

Metrics are created according to movements in the phases. A metric can be based on movement in a specific phase, across multiple phases, and across all the phases. Metrics may be derived from other metrics (e.g., pelvis sway may be the movement of the pelvis stance shown as a percentage of a stance width).

The system turns metrics and other time series data of the user and related equipment (e.g. bat, club, ball) into scores. Scores may be filtered according to the users' desire to work on certain aspect of the action, such as the backswing on a golf swing. Scores may be derived based on other performances by the current user or another user, or a combination of users. Scores can be combined into a configurable hierarchy. This provides the opportunity to prioritize feedback and include different models or methods of coaching.

The system supports different techniques for performing the same skill. The system supports rating the quality of a performance, which can be determined by comparing a current performance and an “optimum” performance.

The optimum performance may be based on individual performances from the user, individual performances from other users, or combinations of performances from individual users or multiple users. The combinations of performances may be grouped according to ability, gender, other biometrics, or combinations thereof. The optimum performance may be defined according to a combination of metrics or sparse geometric data.

The system provides customized feedback to the user by quantifying the difference in their performances and the optimum performances selected by a coach or the user herself. This provides an opportunity to prioritize feedback and include different models or methods of coaching.

The system is adaptable and can be modified to accommodate additional activities, user profile, and user biometrics.

The system uses the user biometrics (e.g., flexibility, somatotype, injury history, and age) to further evolve.

For ease of understanding, the game of golf is used throughout the disclosure to demonstrate various aspects of the claimed invention. However, the same concepts are applicable to other physical activities.

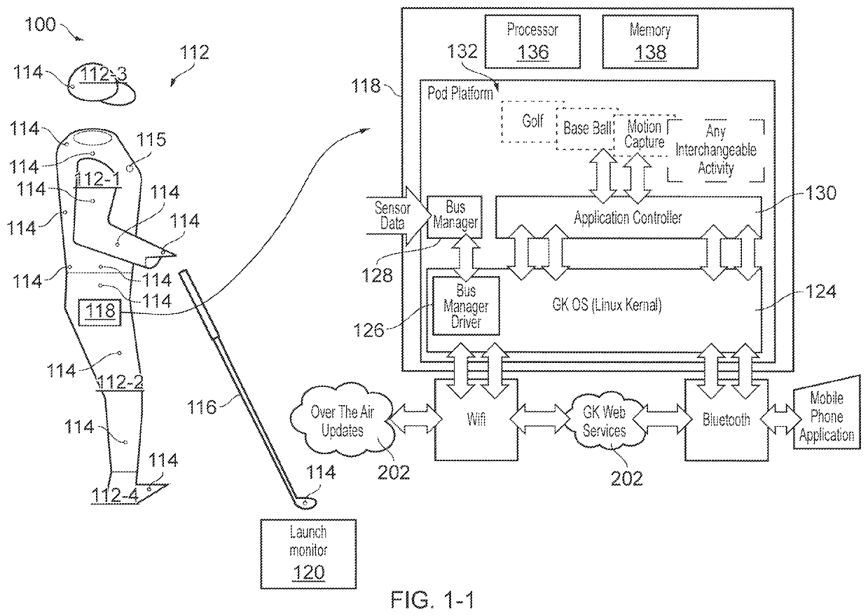

FIG. 1-1 is a block diagram of a motion sensing system 100 in some examples of the present disclosure. Motion sensing system 100 is customizable and configurable according to the action undertaken by a user, as well as the profile and the biometrics (including anthropometry) of the user.

Motion sensing system 100 includes a motion capture suit 112 with motion tracking sensors 114 that directly measure movements of the user wearing suit 112 during an activity to be analyzed. Motion capture suit 112 may be one piece or may include multiple sections, such as an upper body section or shirt 112-1, a lower body section or pants 112-2, a cap or hood 112-3, and socks or insoles 112-4. Motion capture suit 112 may be elastic and relatively tight fitting to minimize shifting of motion tracking sensors 114 relative to the body. Motion tracking sensors 114 are located in or on motion capture suit 112 to measure movement of specific body parts. Each motion tracking sensor 114 may include a combination of accelerometer, gyroscope, and magnetometer, whose raw data outputs may be processed to determine the position, orientation, and movement of the corresponding body part. The raw data outputs include accelerations (m/s²) along the measurement axes of the accelerometer, angular velocity (rad/s) about the measurement axes of the gyroscope, and magnetic field vector components (Gauss) along the measurement axes of the magnetometer.

Motion sensing system 100 may include one or more biofeedback sensors 115 that measure physiological functions or attributes of the subject such as the user's heart rate, respiration, blood pressure, or body temperature. Biofeedback sensors 115 may also provide more data for generating metrics that can be displayed alongside an avatar of the user. For example, a pressure insole in the user's shoe can measure the timing and amount of weight transfer from one foot to another or between the ball and toe of the subject's foot during the measured activity.

The user may employ a piece of equipment 116 during the measured activity. Equipment 116 may, for example, be sporting equipment such as a golf club, a tennis or badminton racket, a hockey stick, a baseball or cricket bat, a ball, a puck, or a shuttle that the subject uses during a measured sports activity. Equipment 116 may alternatively be a tool, exercise equipment, a crutch, a prosthetic, or any item that a subject is being trained to use. One or more motion tracking sensors 114 may be attached to equipment 116 (also referred to as “sensor 116,” such as a club sensor 116) to measure the position, orientation, or movement of equipment 116.

Motion sensing system 100 may include an electronic device 120 that provides additional motion data of the subject or equipment 116, such as a golf launch monitor that provides swing speed, ball speed, and ball spin rate.

A sensor controller 118, also known as a “pod,” is attached to or carried in motion capture suit 112. Pod 118 has wired or wireless connections to sensors 114 and 115. Pod 118 processes the raw motion data from sensors 114 to produce geometric data for a skeletal model and metrics calculated from a combination of the raw motion data and the geometric data. Pod 118 transmits the geometric data, the metrics, and the biofeedback data to an app 134 on a smart device via Bluetooth or a physical connection during or after the measured activity. The smart device may be a smart phone, a tablet computer, a laptop computer, or a desktop computer. App 134 generates scores from the geometric data and provides visual feedback in the form of an avatar that shows the movement of the user.

For hardware, pod 118 includes processor 136 and memory 138 for executing and storing the software. Pod 118 also includes a RS-485 data bus transceiver (not shown) for communicating with sensors 114 and 115, a Wi-Fi transceiver (not shown) for communicating with a wireless network to access the Internet, and a Bluetooth transceiver (not shown) for communicating with app 134 on the smart device.

For software, pod 118 includes an operating system (OS) 124 with a bus manager driver 126 executed by the processor, a bus manager 128 executed by the data bus transceiver, and an application controller 130 that runs on the OS. Application controller 130 includes a number of activity executables 132 that detect and analyze actions of different activities (e.g., a golf swing for golf, a bat swing for baseball, and a groundstroke for tennis).

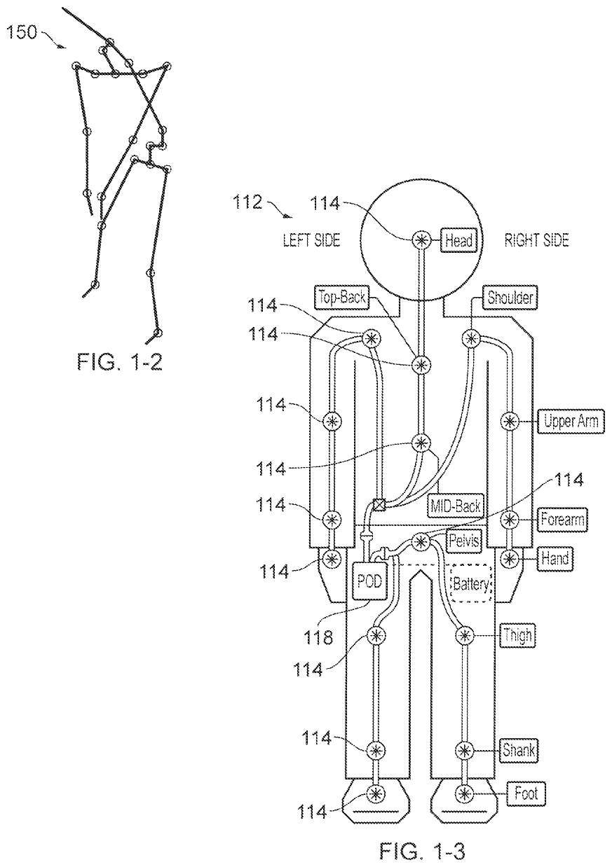

FIG. 1-2 illustrates a skeletal model 150 for generating the avatar in some examples of the present disclosure. Skeletal model 150 is a hierarchical set of joint nodes and limb segments that link the joint nodes. Each limb segment represents a bone, or a fixed bone group, in the human body. The highest joint node in the hierarchy is the root node. In a chain of joint nodes, the joint nodes closer to the root node are higher in the hierarchy than joint nodes further from the root node. Movements of skeletal model 150 are represented by movements of the joint nodes. Assuming the limb segments are rigid bodies, movement of any joint node is represented by a translation (e.g., a vector) and a rotation (e.g., a quaternion) of the joint node, where the rotation of the joint node determines the orientation of the limb segment extending from the joint node. Movement of the root node controls the position and orientation of the skeleton model in a three-dimensional space. Movement of any other joint node in the hierarchy is relative to that node's parent. In particular, all of the descendant joint nodes from the root node form an articulated chain, where the coordinate frame of a child node is always relative to the coordinate frame of its parent node.

To calculate the relative orientation between each node and its corresponding limb segment, the user is required to stand in two known poses: neutral and diving, for a short duration while node orientations are recorded. In the calibration, for the neutral pose, the user stands vertically with their arms by sides. For the diving pose the user stands with hand together, arms horizontal out in front. The movement of the arms from neutral to diving pose allows the system to determine the compass direction the user was facing when the neutral pose was recorded. The neutral pose allows the system to calculate the relative orientation between the sensor nodes and their associated limb segments. A secondary benefit of the diving pose is to correct wrist flexion inaccuracy in the user's actual neutral pose.

FIG. 1-3 illustrates motion capture suit 112 and the approximate locations of motion sensor 114 in some examples of the present disclosure. Motion sensors 114 are inertial measurement unit (IMU) sensors. Motion sensors 114 are placed at strategic locations over the body to track each major limb segment while minimizing movements due to contractions of underlying muscle mass. For each limb segment, the corresponding motion sensor 114 is generally located near the distal end of and on the outer (lateral) surface of the limb segment. For example, a motion sensor 114 for the arm is located near the distal end of the arm toward the elbow.

FIG. 2 is a swimlane diagram demonstrating how pod 118 is configured in some examples of the present disclosure. In step 1, upon startup, application controller 130 requests for the system configuration of pod 118 from a provider 202 of motion sensing system 100 over the Internet. The system configuration identifies sensors, activity executables, and other hardware and software components that the user is authorized to use (e.g., by subscription). Application controller 130 may connect to the Internet through the Wi-Fi or Bluetooth. In step 2, provider 202 sends the system configuration to application 130 to verify and enable the authorized hardware and software for the user.

In step 3, an interactive app 134 (FIGS. 1 and 2) on a smart device requests to connect with application controller 130 over Bluetooth. The smart device may be a laptop, a smart phone, or a tablet computer. In steps 4 and 5, application controller 130 and app 134 exchange handshake messages to establish a connection. In step 6, app 134 sends a user-selected activity to application controller 130. In step 7, app 134 sends a new skill model of the user-selected activity to application controller 130. The skill model identifies a particular action or a task (e.g., training) that the user will perform. The task may be a repetition or a combination of actions assigned by a coach.

In step 8, when an activity executable 132 (FIG. 1) for the activity has not been previously downloaded, application controller 130 requests the activity executable 132 from the cloud, i.e., from provider 202 over the Internet. Activity executable 132 includes a suit configuration (i.e., sensor configurations) for the user-selected activity and code for detecting actions, recognizing phases in the actions, and extracting metrics the phases. In step 9, provider 202 sends activity executable 132 to application controller 130 over the Internet.

In step 10, application controller 130 requests bus manager driver 126 to open a connection to motion capture suit 112. In step 11, bus manager driver 126 requests bus manager 128 to enable motion capture suit 112. In step 12, bus manager 128 informs bus manager driver 126 that motion capture suit 112 has been turned on. In step 13, bus manager driver 126 informs application controller 130 that motion capture suit 112 has been turned on. In step 14, application controller 130 sends the suit configuration for the activity to bus manager driver 126. In step 15, bus manager driver 126 forwards the suit configurations to bus manager 128, which configures motion sensors 114 accordingly. In step 16, bus manager 128 sends a ready status, suit diagnostic information, and an identification (ID) of motion capture suit 112 and sensors 114, 115 to bus manager driver 126. In step 17, bus manager driver 126 forwards the ready status, the suit diagnostic information, and the suit and sensor IDs to application controller 130. In step 18, application controller 130 sends the suit diagnostic information and the suit and sensor IDs to provider 202 for recording keeping and maintenance purposes.

In step 19, application controller 130 informs app 134 that application controller 130 is ready to capture motion data of the new activity. In step 20, application controller 130 runs activity executable 132 for the activity. In step 21, app 134 instructs application controller 130 to begin acquiring raw motion data from motion sensors 114 in motion capture suit 112 and on sports equipment 116. In some examples, application controller 130 also begin acquiring motion data from electronic device 120. In step 22, application controller 130 generates sparse geometric data streams from the raw data streams. Whereas the raw data streams contain motion data at a regular interval (e.g., 1,000 samples per second), the sparse data streams contain motion data when there is sufficient change from a prior value or sufficient time has passed from when the last value was recorded. In other words, one or more portions of a sparse data stream may contain motion data at irregular intervals. Also in step 22, activity executable 132 recognizes the action and its phases from the raw data streams and the sparse geometric data streams, extracts metrics from the phases, and sends the sparse geometric data streams and the metrics to app 134, which generates scores and the appropriate visual feedback to the user. The visual feedback may be an avatar, which is generated with skeletal model 150 in FIG. 1-2, illustrating the movement of the user.

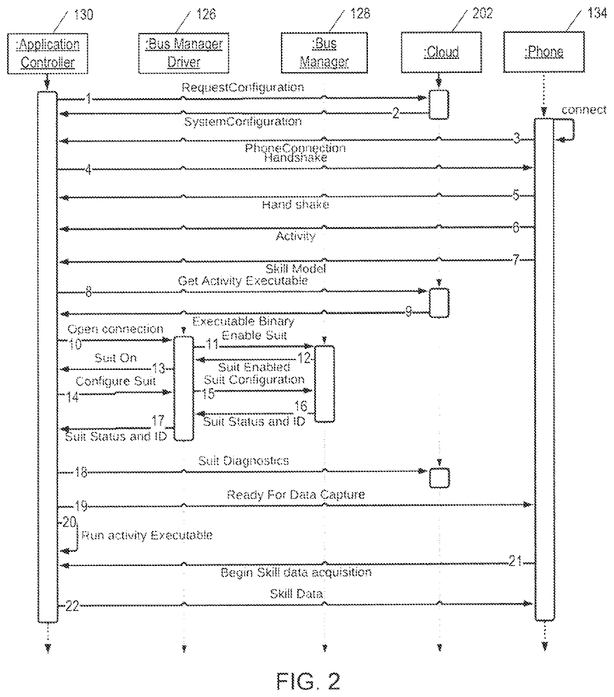

FIG. 3 is a flowchart of a method 300 illustrating the operations of pod 112 (FIG. 1), more specifically application controller 130 (FIGS. 1 and 2) and activity executable 132 (FIGS. 1 and 2), in some examples of the present disclosure. Method 300, and any method described herein, may be implemented as instructions encoded on a computer-readable medium that is to be executed by a processor a computing system. Method 300, and any method described herein, may include one or more operations, functions, or actions illustrated by one or more blocks. Although the blocks are illustrated in sequential orders, these blocks may also be performed in parallel, and/or in a different order than those described herein. In addition, the various blocks may be combined into fewer blocks, divided into additional blocks, and/or eliminated based upon the desired implementation. Method 300 may start with block 302.

In block 302, application controller 130 receives profile and biometrics of the user. Alternatively, application controller 130 retrieves from memory the user profile and the user biometrics. The user profile includes gender, date of birth, ethnicity, location, skill level, recent performance data, and health information. The user may input her profile through app 134 (FIGS. 1 and 2), which transmits the information to application controller 130. The user biometrics include passive range of movement at each joint, active range of movement at each joint, strength indicator, anthropometric measurements, resting heart rate, and breathing rates. Biometric measurements may be taken at point of sale of pod 118 and recorded in the memory of the pod 118. Alternatively, the user may input her biometrics through app 124, which transmits the information to application controller 130. The user biometrics may be periodically updated by using app 134 or through provider 202 over the Internet. Block 302 may be followed by block 304.

In block 304, application controller 130 receives the user-selected activity and action from app 124. This corresponds to steps 6 and 7 of method 200 in FIG. 2. Alternatively, application controller 130 retrieves the last selected activity and action from memory.

As mentioned previously, an action may be a physical skill, a technique of a skill, a variation of a technique, or a pose. A skill is defined as a movement that is part of an overarching activity (movement sequence, relate to medical or medical condition health) or a sport. For example, the golf swing is at the core of the game of golf. Nearly all golf swings have some aspects in common (e.g. using a club, a backswing, a downswing, a follow-through), which may be used to specify how to analyze a skill and, in particular, how to detect and analyze the skill to provide feedback.

The skill can be further specified according to a technique of the skill, the equipment being used, and a variation of the technique. For golf, the technique may be a type of shot, such as a drive, approach, chip, or putt, the equipment may be a type of golf club, such as a driver, 3 wood, or 7 iron, and the variation of the technique may be a shot shaping, such as straight, fade, draw, high, or low. For tennis, the technique may be a type of groundstroke, such as a forehand, backhand, volley, or serve, the equipment may be a type or size of tennis racket, such as stiffness, weight, or head size, and the variation of the technique may be a ball spin, such as topspin, flat, or slice. Specifying such information allows activity executable 132 to better identify when a user completes a skill.

Block 304 may be followed by block 306.

In block 306, application controller 130 configures motion sensors 114 for detecting the action. This corresponds to step 14 in method 200 of FIG. 2. For example, application controller 130 turns on a number of motion sensors 114 and sets their sampling rates for detecting the action. As described above, motion sensors 114 may be part of motion capture suit 112 (FIG. 1) and equipment 116. Block 306 may be followed by block 308.

In block 308, application controller 130 receives time series of raw motion data (raw data streams) from corresponding motion sensors 114. Application controller 130 may also receive time series of biofeedback data (biofeedback data stream) from biofeedback sensor 115. Application controller 130 may further receive time series of additional motion data (additional motion data stream) from electronic device 120 (FIG. 1). Block 308 may be followed by block 310.

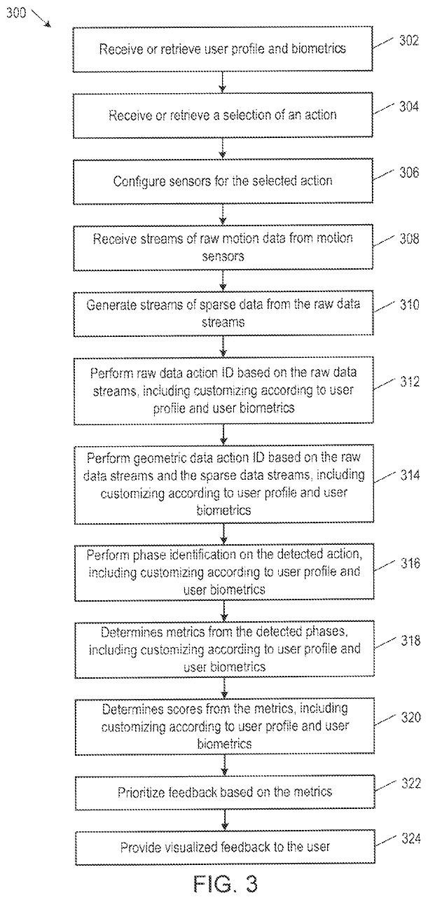

In block 310, application controller 130 generates time series of sparse geometric data (sparse geometric data streams) from the raw data streams. An implementation of block 309 is described later in reference to FIG. 4. Block 310 may be followed by block 312.

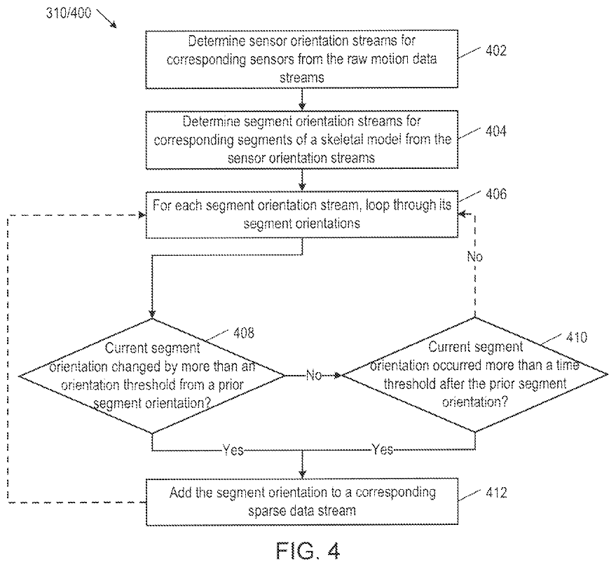

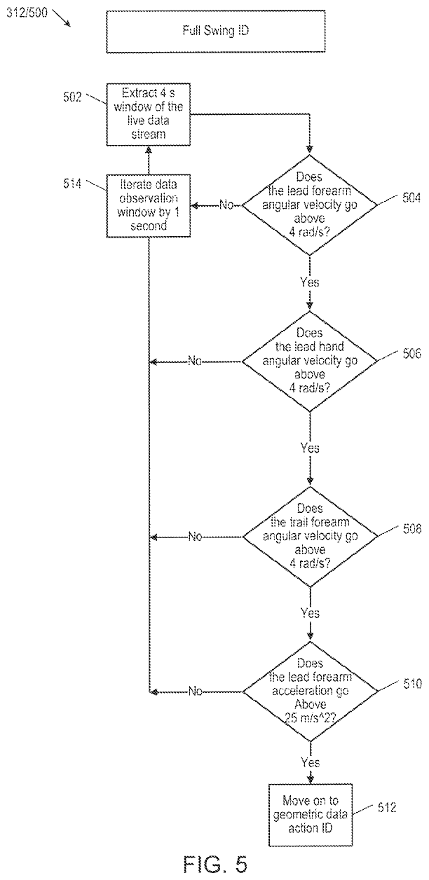

In blocks 312 and 314, activity executable 132 performs action identification (ID). In block 312, activity executable 132 performs raw data action ID to detect the action in time windows of the raw data streams. Activity executable 132 may use different thresholds and different motion sensors 114 or combinations of motion sensors 114 to identify different skills and different techniques. As this process uses raw motion data, it allows faster data processing than using more processed (fused) data. To improve detection, activity executable 132 may also use biofeedback data stream from biofeedback sensor 115 and the additional motion data stream from electronic device 120. Activity executable 132 may modify the identification process based on the user profile and the user biometrics received or retrieved in block 302. Implementations of block 312 are described later in reference to FIGS. 5 and 6. Block 312 may be followed by block 314.

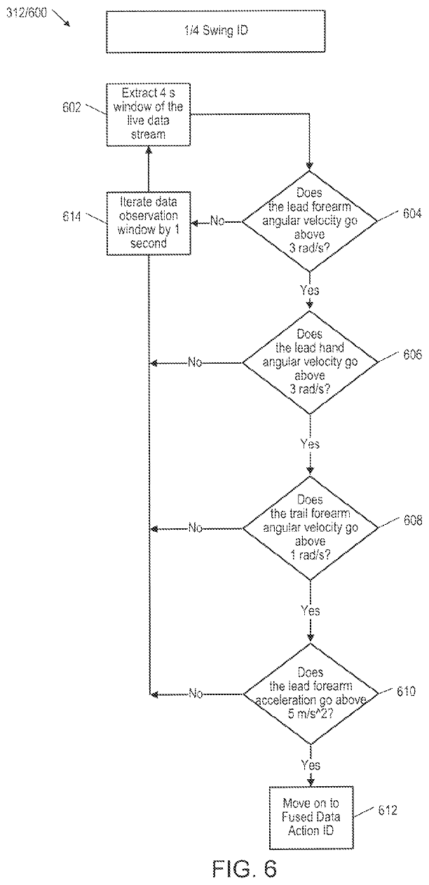

In block 314, activity executable 132 performs geometric data action ID to detect the action in the time windows identified in the raw data action ID. Activity executable 132 performs geometric data action ID based on the sparse geometric data streams in the identified time windows. To improve detection, activity executable 132 may also use the raw data streams from motion sensor 114, biofeedback data stream from biofeedback sensor 115 and the additional motion data stream from electronic device 120. Activity executable 132 may modify the identification process based on the user profile and the user biometrics received or retrieved in block 302. An implementation of block 311 is described later in reference to FIG. 7. Block 311 may be followed by block 316.

In block 316, activity executable 132 performs phase ID to detect phases of the detected action in the time windows identified in the geometric data action ID. Activity executable 132 performs phase ID based on the sparse geometric data streams in the identified time windows. To improve detection, activity executable 132 may also use the raw data streams from motion sensor 114, biofeedback data stream from biofeedback sensor 115 and the additional motion data stream from electronic device 120. Activity executable 132 may modify the phase identification based on the user profile and the user biometrics. Implementations of block 316 are described later in reference to FIGS. 8, 9, 10, 11-1, and 11-2. Block 316 may be followed by block 318.

In block 318, activity executable 132 determines metrics from the phases identified the phase ID. Activity executable 132 extras the metrics from on the sparse geometric data in the identified phases. Activity executable 132 may also extract the metrics from the raw data stream from motion sensor 114, biofeedback data stream from biofeedback sensor 115, and the additional motional data stream from electronic device 120. Activity executable 132 may modify the metrics being detected based on the user profile and the user biometrics. Details of block 318 are described later. Block 318 may be followed by block 320.

In block 320, app 124 determines scores based on the metrics received from pod 118. App 124 may modify the scoring based on the user profile and the user biometrics and according to preferences of the user or a coach. Details of block 320 are described later. Block 320 may be followed by block 322.

In block 322, app 124 prioritizes feedback by applying weights to the scores, summing groups of the weighted scores to generate group summary scores, applying weights to the group summary scores, summing supergroups of the weighted group summary scores to generate supergroup summary scores, and generating a hierarchical structure based on the group summary scores and the supergroup summary scores. Details of block 322 are described later. Block 322 may be followed by block 324.

In block 324, app 124 uses the sparse geometric data streams of the detected action in identified windows to create and animate the avatar. App 124 may use the hierarchical structure of the scores to creates a visual comparison between the avatar and an optimum performance. App 124 may enhance the visual comparison by indicating angle, distance, speed, or acceleration notations based on the scores to highlight areas of interest. App 124 may also playback media based on the hierarchical structure of the scores. Details of block 324 are described later.

Motion Capture Data (Time Series Data Compression)

Motion sensors 114 output time series of raw motion data. Each motion sensor 114 is located near the distal end of the corresponding limb segment to provide the orientation, velocity, and acceleration of the limb segment. When the segment orientation is represented by quaternions at high frequency, it would consume large amount of processor time and memory in pod 106. Application controller 130 processes the raw data streams, as it is generated by motion sensors 114, into sparse geometric data streams for each limb segment. To compress the data into a more compact form with minimal loss of information, application controller 130 only adds a new orientation of a limb segment to the corresponding sparse geometric data stream if the new orientation differs by more than an orientation threshold (e.g., one degree) from the previous orientation in the stream or if the new orientation has a timestamp greater than a time threshold (e.g., one second) later than the previous orientation in the stream. These sparse geometric data streams can be quickly interpolated at any time and put together to form an accurate skeletal model or other time-varying quantities for processing. The orientation and time thresholds are customizable for different actions or compression requirements.

FIG. 4 illustrates a method 400 for implementing block 310 (FIG. 3), which generates sparse geometric data streams, in some examples of the present disclosure. Method 400 may begin in block 402.

In block 402, application controller 130 determines time series of sensor orientations (sensor orientation streams) for the corresponding motion sensors 114 from their raw data streams. For example, application controller 130 converts the raw data stream from the accelerometers, gyroscopes, and magnetometers of each motion sensor 114 into a sensor orientation stream. Block 402 may be followed by block 404.

In block 404, application controller 130 determines streams of segment orientations (segment orientation stream) for the corresponding limb segments in the skeletal model from the sensor orientation streams. Block 404 may be followed by block 406.

In block 406, application controller 130 loops through the segment orientations of each segment orientation stream. Block 406 may be followed by block 408.

In block 408, application controller 130 determines if the current segment orientation in the segment orientation stream changed by more than the orientation threshold from a prior segment orientation. If so, block 408 may be followed by block 412. Otherwise block 408 may be followed by block 410.

In block 410, application controller 130 determines if the current segment orientation occurred more than the time threshold after the prior segment orientation from their timestamps. If so, block 408 may be followed by block 412. Otherwise block 410 may loop back to block 406 to select the next segment orientation in the segment orientation stream until all the segment orientations have been processed.

In block 412, application controller 130 adds the current segment orientation to the corresponding sparse geometric data stream. Block 412 may loop back to block 406 to select the next segment orientation in the segment orientation stream until all the segment orientations have been processed.

Action Recognition

Given the user-selected action (a skill, a technique of the skill, a variation of the technique, or a pose) and the system configuration, application controller 130 can recognize when the action is performed. Application controller 130 can use raw data streams from motion sensor 114 to detect that the action has been performed. This is referred to as “raw data action ID” and examples for a golf drive of different sizes are described below in reference to FIGS. 5 and 6. Application controller 130 can detect different skills and techniques by using different thresholds and different motion sensors 114, or combinations of motion sensors 114. Application controller 130 can more quickly detect the action by using raw data streams than using processed (fused) data streams, which would take longer to generate and process.

Application controller 130 can customize the raw data action ID to the user according to her profile and biometrics. For example, a young elite golfer (identified using handicap) may perform a golf swing in a concise and fast manner, whereas an elderly novice golfer may perform the same technique more slowly and with a less clear start and end point. In this example, application controller 130 adapts to the given user. Such adaptations may include a shorter or longer data search window (e.g. 4 sec. for the elite golfer and 10 sec. for the elderly golfer) and higher or lower raw data action recognition thresholds (e.g. 5 rad.s-1 for the left hand gyro for the elite golfer and 3 rad.s-1 for the elderly golfer). Female golfers may typically hit less hard than male golfers and so the raw data thresholds may be reduced. Children are likely to hit less hard than adults and so the raw data thresholds may be lowered for them. Taller golfers (or golfers with longer limbs) are likely to achieve higher linear accelerations and therefore may use higher acceleration thresholds. Golfers with disabilities (or injuries) may require more specific raw data thresholds or even use alternative sensors 114 according to their requirements. Left and righthanded golfers may require the use of different sensors 114. Variants of a technique may require different motion sensors 114 or raw data thresholds (e.g. overarm vs sidearm baseball pitching, front-on vs side-on cricket bowling). FIGS. 5 and 6 show how the raw data thresholds may change for different sizes of golf swing.

If an additional sensor 115 is added to motion sensing system 100, such as a proximity sensor, a heat sensor (thermometer), or a water sensor, information from such a sensor may be used to refine the raw data action ID or observe additional phases (see below). For example, in team sports, application controller 130 may use the proximity sensor to detect an approaching opponent and trigger the raw data action ID earlier. In swimming or diving, a water sensor may be used to detect entry into the water. In industrial use or use in firefighting, a heat sensor may be used to trigger the raw data action ID according to a temperature threshold.

For cases where it is desirable to observe more than one action (technique) at a time, multiple raw data action ID methods are run concurrently to detect the desired actions (techniques and variations of techniques). For example, motion sensing system 100 may be used for gymnastics where the user may perform many techniques one after another, such as in a floor routine.

Complimentary executables may perform raw data action ID methods to detect additional skills performed by the user that are not part of the activity executables 132 installed on pod 118 but are related to the activity. This provides the opportunity to identify that the user is performing the additional skills and offer the appropriate activity executables 132 to download to pod 118. Application controller 130 may automatically download the complimentary executables that detect the additional skills. For example, the user may be practicing cricket batting using an activity executable 132 and a complimentary executable also detects the user is running. In response, the complimentary executable causes application controller 130 to triggers app 134 to offer the user an activity executable 132 for running.

FIG. 5 illustrates a method 500 for implementing block 312 (FIG. 3), which performs raw data action ID, in some examples of the present disclosure. In particular, method 500 detects a full swing. Method 500 may begin in block 502.

In block 502, activity executable 132 extracts a window in time (e.g., 4 sec.) of the live raw data streams from motion sensor 114. Block 502 may be followed by block 504.

In block 504, activity executable 132 determines if the angular velocity of a first limb segment in the skeletal model (e.g., the lead forearm) exceeds a first angular velocity threshold (e.g., 4 rad/s) in the window. If so, block 504 may be followed by block 506. Otherwise block 504 may be followed by block 514.

In block 506, activity executable 132 determines if the angular velocity of a second limb segment in the skeletal model (e.g., the lead hand) exceeds a second angular velocity threshold (e.g., 4 rad/s) in the window. If so, block 506 may be followed by block 508. Otherwise block 506 may be followed by block 514.

In block 508, activity executable 132 determines if the angular velocity of a third limb segment in the skeletal model (e.g., the trail forearm) exceeds a third angular velocity threshold (e.g., 4 rad/s) in the window. If so, block 508 may be followed by block 510. Otherwise block 508 may be followed by block 514.

In block 510, activity executable 132 determines if the acceleration of the first limb segment (e.g., the lead forearm) exceeds an acceleration threshold (e.g., 25 m/s²) in the window. If so, block 510 may be followed by block 512. Otherwise block 510 may be followed by block 514.

In block 512, when the raw data action ID is passed, activity executable 132 passes the window to geometric data action ID as described later in reference to FIG. 7.

In block 514, activity executable 132 iterates or moves the window by a predefined time (e.g., 1 sec.). Block 514 may loop back to block 502 to perform the raw data action ID on the new window.

FIG. 6 illustrates a method 600 for implementing block 312 (FIG. 3), which performs raw data action ID, in some examples of the present disclosure. In particular, method 600 detects a quarter (¼) swing. Method 600 is similar to method 500 except the various thresholds have been adjusted for the quarter swing. Method 600 may begin in block 602.

In block 602, activity executable 132 extracts a window in time (e.g., 4 sec.) of the live raw data streams from motion sensor 114. Block 602 may be followed by block 604.

In block 604, activity executable 132 determines if the angular velocity of a first limb segment in (e.g., the lead forearm) exceeds a first angular velocity threshold (e.g., 3 rad/s) in the window. If so, block 604 may be followed by block 606. Otherwise block 604 may be followed by block 614.

In block 606, activity executable 132 determines if the angular velocity of a second limb (e.g., the lead hand) exceeds a second angular velocity threshold (e.g., 3 rad/s) in the window. If so, block 606 may be followed by block 608. Otherwise block 606 may be followed by block 614.

In block 608, application controller 130 determines if the angular velocity of a third limb segment in (e.g., the trail forearm) exceeds a third angular velocity threshold (e.g., 1 rad/s) in the window. If so, block 608 may be followed by block 610. Otherwise block 608 may be followed by block 614.

In block 610, application controller 130 determines if the acceleration of the first limb segment (e.g., the lead forearm) exceeds an acceleration threshold (e.g., 5 m/s²) in the window. If so, block 610 may be followed by block 612. Otherwise block 610 may be followed by block 614.

In block 612, when the raw data action ID is passed, activity executable 132 passes the identified window to geometric data action ID as described later in reference to FIG. 7.

In block 614, activity executable 132 iterates or moves the window by a predefined time (e.g., 1 sec.). Block 614 may loop back to block 602 to perform the raw data action ID on the new window.

After raw data action ID, activity executable 132 can use the sparse geometric data streams and optimally the raw data streams to detect the action has been performed. This is referred to as “geometric data action ID” and the example of a golf drive is described below in reference to FIG. 7. The geometric data action ID may be customized for each technique of interest and further tailored to the user according to her profile and user biometrics as described above for the raw data action ID.

FIG. 7 illustrates a method 700 for implementing block 314 (FIG. 3), which performs geometric data action ID, in some examples of the present disclosure. In particular, method 700 detects a swing. Method 700 may begin in block 702.

In block 702, activity executable 132 selects a new window in time (e.g., 4 sec.) that passed the raw data action ID and processes the sparse geometric data streams and the raw data streams in the new window. Block 702 may be followed by block 704.

In block 704, application controller 130 determines the approximate time of contact (ATC) by the club with the golf ball. For example, activity executable 132 determines the most recent time the largest spike in the acceleration of a first limb segment (e.g., the lead hand) occurred in the window. Block 704 may be followed by block 706.

In block 706, activity executable 132 determines the approximate top of the backswing (ATB). For example, activity executable 132 determines if, before the ATC, the angular velocity of a second limb segment (e.g., the lead forearm) exceeded a first angular velocity threshold (e.g., 0.4 rad/s) in the window. If no, block 706 may be followed by block 708. Otherwise block 706 may be followed by block 710.

In block 708, activity executable 132 determines if, before the ATC, the angular velocity of the first limb segment (e.g., the lead hand) exceeded a second angular velocity threshold (e.g., 0.4 rad/s) in the window. If so, block 708 may be followed by block 710. Otherwise block 708 may loop back to block 702 to process a new window that passed the raw data action ID.

In block 710, activity executable 132 determines if, from the ATC to 0.5 sec. after the ATC, the first and the second limb segments (e.g., the lead hand and the lead forearm) were within 10 degrees of being pointed straight down to the ground. If so, block 710 may be followed by block 712. Otherwise block 710 may loop back to block 702 to process a new window that passed the raw data action ID.

In block 712, activity executable 132 determines if the time of the first limb segment (e.g., the lead hand) being pointed most straight down to the ground occurred more than 0.01 sec. after ATB. If so, block 712 may be followed by block 714. Otherwise block 712 may loop back to block 702 to process a new window that passed the raw data action ID.

In block 714, activity executable 132 determines if, between the ATB and the most pointed down time of the first limb segment (e.g., the lead hand), the average angular velocity of both the second limb segment and a third limb segment (e.g., both of the forearms) were within a third angular velocity threshold (e.g., 20 deg/s or 0.3490658504 rad/s) of each other. If so, block 714 may be followed by block 716. Otherwise block 714 may loop back to block 702 to process a new window that passed the raw data action ID.

In block 716, activity executable 132 proceeds to phase recognition as described later in reference to FIGS. 8, 9, 10, 11-1, and 11-2.

Phases and Critical Moments Therein

Each skill or technique can be divided into a number of phases. The phases may vary according to the skill or technique being performed. They may also vary according to the desires of the coach and the level of performer. For example, a skill is commonly divided into preparation, action, and recovery phases. However, a more experienced performer may wish a greater breakdown of the performance than a novice. For example, the golf drive is divided into the following ten phases.
1) Address—Initial pose for lining up with the golf ball
2) Mid-Backswing—When the club is horizontal in the backswing
3) Lead Arm Parallel—When the lead arm is horizontal in the backswing
4) Top of Backswing—When the club reaches its maximum angle in the backswing
5) Transition—When the lead arm is horizontal in the downswing
6) Mid-Downswing—When the club is horizontal in the downswing
7) Contact—When the club strikes the ball
8) Mid-Follow through—When the club is horizontal in the follow through
9) Trail Arm Parallel—When the trail arm is horizontal in the follow through
10) Finish—When the club reaches its maximum angle in the follow through

Some phases (e.g., address and contact) must be present for application controller 130 to acknowledge and analyze a full golf swing, while other phases (e.g., lead arm parallel and transition) may be absent. For example, a golfer may perform a full golf swing but their limited range of movement in the backswing might mean that their lead arm does not reach a point where it is horizontal to the ground and hence the lead arm parallel phase would not be available for analysis.

Phase Recognition

Once a potential action (skill, technique, or phase) is recognized in a given window from the raw and the geometric data action ID, activity executable 132 searches the window for the phases specified for the action. The order and method of finding each of the phases must be specified for each action. If a piece of equipment 106, such as a golf club, is essential to the process, the phase recognition is simplified when the equipment also has a motion sensor 114 on it. If this is not possible, or has not happened, activity executable 132 can predict the location and orientation of the equipment based on the closest limb segment or a combination of the closest limb segments.

It is possible that the user has moved in a way that passes the raw and the geometric data action ID but the user was not actually performing the action of interest. In this case, the phase recognition may also act as a backup to verify the action has been actually performed. That is, if a critical phase cannot be found in the window, activity executable 132 assumes the window does not contain the skill or technique of interest.

As with the raw and the geometric data action ID, activity executable 132 can customize the phase recognition process for the user according to her profile and biometrics. For example, some phases may not be required for a less proficient performer or may even be removed altogether in order to reduce the complexity of analysis and feedback. Variants of a technique may require an additional sensor or different thresholds (e.g. overarm vs sidearm baseball pitching, front-on vs side-on cricket bowling). If an additional sensor 115 or 116 is added to motion sensing system 100, such as an equipment sensor, proximity sensor, heat sensor, water sensor, or temperature sensor, information from these sensors may be used to refine or observe additional phases. In team sports, a proximity sensor 115 may be used to detect an approaching opponent and add an earlier phase to the action. In swimming or diving, a water sensor 115 may be used refine the point of entry into the water or stroke detection. In golf, a club sensor 116 or launch monitor 120 may be used to refine detection of the point of impact. Knowledge of the club being used means its length and stiffness may be determined from its manufacturer. This information can refine a mathematical model of the club (used when a sensor 116 is not on the club), which in turn can refine the accuracy of the phase identification of the golf swing. Knowledge of handedness will determine the sensors used (e.g., right or left hand as the lead hand). Knowledge of the ability (or disability) of the golfer may mean higher or lower thresholds are set, such as in the raw and the geometric data action ID, to determine the onset or start of a movement.

In turn this knowledge of the user and the skeletal model allow detailed comparison of movements within and between users through synchronized side by side and overlays of performances.

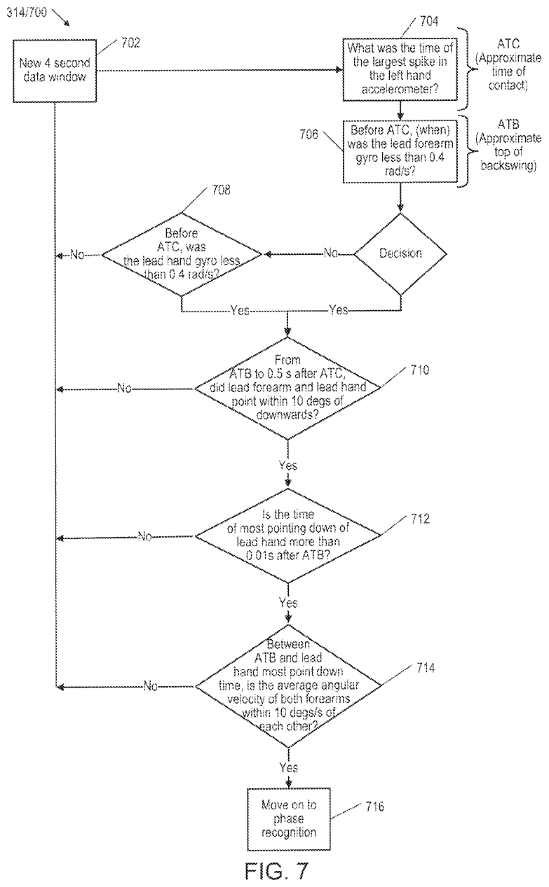

FIG. 8 is a block diagram illustrating a method 800 for phase ID of a generic action in some examples of the present disclosure. Method 800 recognizes five phases by performing the following identifications in order: start of movement ID 802, preparation phase ID 804, action phase ID 806, recovery phase ID 808, and end of movement ID 810.

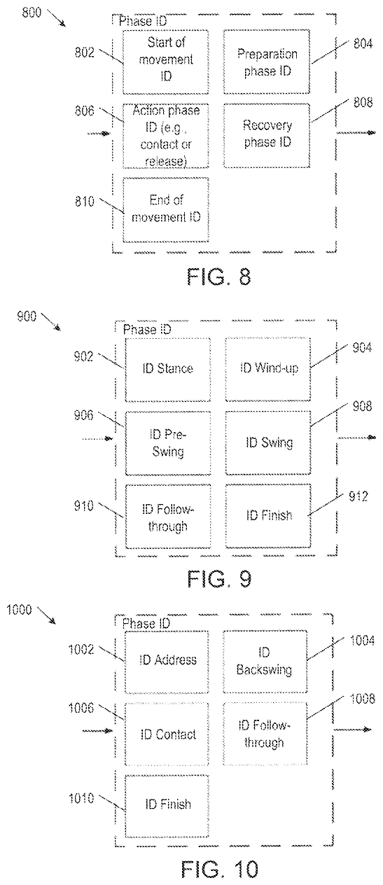

FIG. 9 is a block diagram illustrating a method 900 for phase ID of a baseball batting action in some examples of the present disclosure. Method 900 recognizes six phases by performing the following identifications in order: stance ID 902, windup ID 904, pre-swing ID 906, swing ID 908, and follow-through ID 910, and finish ID 912.

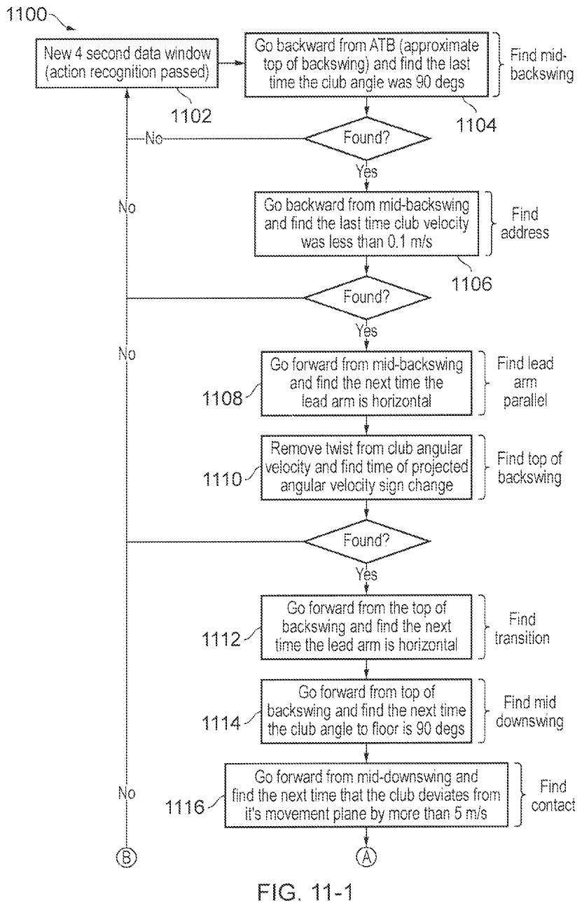

FIG. 10 is a block diagram illustrating a method 1000 for phase ID of a golf swing in some examples of the present disclosure. Method 1000 recognizes five phases by performing the following identifications in order: address ID 1002, backswing ID 1004, contact ID 1006, follow-through 1008, and finish ID 1010.

As described above, a more experienced performer may wish a greater breakdown of the performance than a novice. FIGS. 11-1 and 11-2 is a flowchart of a method 1100 for phase ID that recognizing ten phases of a golf swing in some examples of the present disclosure. Method 1100 may begin in block 1102.

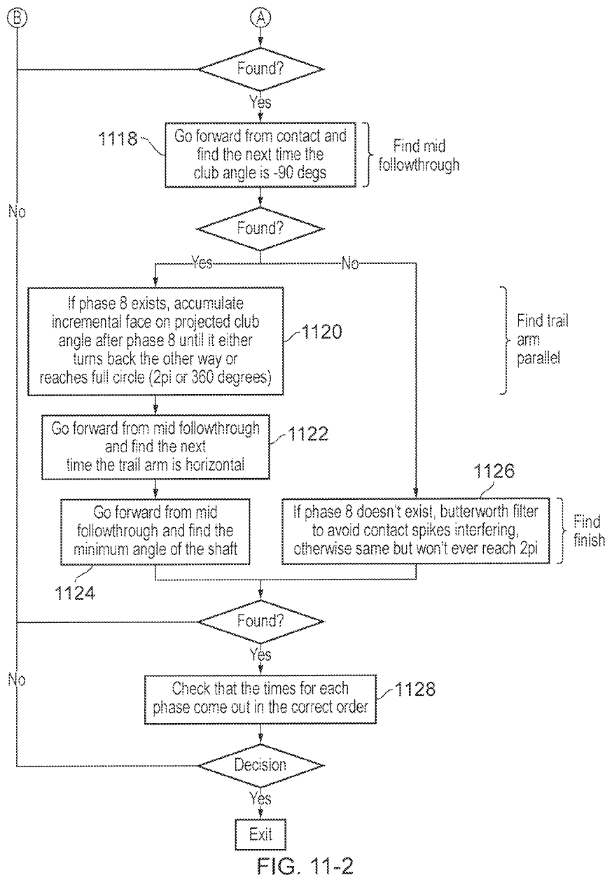

Referring to FIG. 11-1, in block 1102, activity executable 132 selects a new window in time (e.g., 4 sec.) that passed the geometric data action ID and processes the sparse data streams and optionally the raw data streams in the new window. Block 1102 may be followed by block 1104.

In block 1104, activity executable 132 goes backward in time from the ATB (approximate top of backswing) to find the most recent time the club angle was at 90 degrees. The club angle may be from processed (fused) data derived from raw data generated by sensor on the club or derived from a mathematical model of the club. If such club angle cannot be found, block 1104 may loop back to block 1102 to select and process a new window. If such club angle is found, activity executable 132 has located the mid-backswing phase and block 1104 may be followed by block 1106.

In block 1106, activity executable 132 goes backward in time from the mid-backswing determined in block 1104 to find the most recent time the club velocity was less than 0.1 m/s. The club velocity may be from processed (fused) data derived from raw data generated by a sensor on the club or derived from a mathematical model of the club. If such club velocity cannot be found, block 1106 may loop back to block 1102 to select and process a new window. If such club velocity is found, activity executable 132 has found the address phase and block 1106 may be followed by block 1108.

In block 1108, activity executable 132 goes forward in time from the mid-backswing determined in block 1104 to find the next time a first limb segment (e.g., the lead arm) is horizontal. If such lead arm orientation is found, activity executable 132 has found the lead arm parallel phase. Block 1108 may be followed by block 1110.

In block 1110, activity executable 132 remove the twist from the club angular velocity to find time when the projected angular velocity changes sign. If such time is found, activity executable 132 has found the top of the backswing. When one of the above two conditions cannot be found, block 1110 may loop back to block 1102 to select and process a new window. When both conditions are found, block 1110 may be followed by block 1112.

In block 1112, activity executable 132 goes forward in time from the top of the backswing to find the next time the lead arm is horizontal. If such lead arm orientation is found, activity executable 132 has found the transition phase. Block 1112 may be followed by block 1114.

In block 1114, activity executable 132 goes forward in time from the top of the backswing to find the next time the club angle is 90 degrees to the floor (e.g., when the club shaft is parallel to the floor). If such club angle is found, activity executable 132 has found the mid-downswing phase. Block 1114 may be followed by block 1116.

In block 1116, activity executable 132 goes forward in time from mid-downswing to find the next time the club deviates from a swing plane by more than 5 m/s. The swing plane may be determined as a plane in a down the field view that passes through the line formed from the club head to the user's upper sternum on the address phase determined in block 1106. If such deviation is found, activity executable 132 has found the contact phase. When any of the above three conditions cannot be found, block 1116 may loop back to block 1102 to select and process a new window. When all three conditions are found, block 1116 may be followed by block 1118 (FIG. 11-2).

Referring to FIG. 11-2, in block 1118, activity executable 132 goes forward in time to find the next time the club angle is at −90 degrees relative to the ground. If such club angle is found, activity executable 132 has found the mid-follow through phase and block 1118 may be followed by block 1120. If such club angle is not found, block 1118 may be followed by block 1126.

In block 1120, activity executable 132 accumulates the angle of the projected club shaft from the time of the mid-follow through until it either turns back the other way or reaches full circle (2 pi or 360 degrees). If the club shaft either turns back or reaches full circle, activity executable 132 has found the trail arm parallel phase. Block 1120 may be followed by block 1122.

In block 1122, activity executable 132 goes forward in time from the mid-follow through to find the next time the trail arm is horizontal. Block 1122 may be followed by block 1124.

In block 1124, activity executable 132 goes forward from mid-follow through to find the minimum angle of the shaft. If such minimum angle of the shaft if found, activity executable 132 has found the finish phase. When any of the above three conditions cannot be found, block 1124 may loop back to block 1102 to select and process a new window. When all three conditions are found, block 1124 may be followed by block 1128.

In block 1126, as the finish phase may not exist, activity executable 132 applies the Butterworth filter to smooth out noise in the sparse data streams to prevent the shock (contact spikes) of the club striking the ball from interfering with phase detection and the actions in blocks 1120 to 1124 are performed again. If the finish phase is again not detected, block 1126 may loop back to block 1102 to select and process a new window. If the finish phase is now detected, block 1126 may be followed by block 1128.

In block 1128, activity executable 132 checks that the times of the detected phases came out in the specified sequential order. If not, block 1128 may loop back to block 1102 to select and process a new window. If so, activity executable 132 has identified all the phases and block 1128 may loop back to block 1102 to select and process a new window.

Metrics

The phase recognition and the time series data in the sparse data streams provide sufficient information to create metrics. Metrics are measurements related to movement that can be used to provide analysis and feedback on the quality of the performance of the skill or the technique. They provide a way to quantify the things that a coach or expert viewing the technique can use to create feedback. Furthermore, they may be used to create automated feedback, as described later in Scores and Prioritized Feedback sections.

Metrics are specific to techniques and their phases, and they can be further customized according user profile and user biometrics. Metrics can be taken at a single point in time during the technique, or multiple points over the technique. For example, in golf, the club speed may only be measured at its peak on the down swing, whereas the lead elbow angle may be measured at every phase of the swing.

Metrics can be configured according to movement or user profile (and biometrics).

Metrics may relate to whole body movement (e.g. center of mass or pressure).

Metrics may relate to single segments or parts of the body (e.g. segment movement or joint angles).

Metrics may be kinetic variables (e.g. peak force, impulse).

Metrics may relate to external equipment or third-party technology (e.g. a golf launch monitor).

Metrics may also be derived from linear and angular velocities, accelerations durations or times (absolute or relative).

Metrics are created according to individual phases, across several phases, or relate to the overall movement (all phases).

Metrics may be derived from other metrics. For example, pelvis sway may be the movement of the pelvis stance shown as a percentage of a stance width.

Metrics may be turned into scores.

For customization according to the user profile or biometrics, elite users may be offered a wider variety of metrics, users with disability or injury may have metrics tailored accordingly (e.g., amputees may not require a certain limb), females may focus on aspects of the technique different from males and so this may determine the order of metrics shown.

Addition of sensors may also facilitate additional metrics. For example, a golf club sensor 116 may allow more detail of the club movement, such as face angle through the swing. Heart rate, blood flow, body temperature, and air temperature may all provide additional metrics according to what each measured.

The metrics for the detected phases are referred to as the current metrics.

Optimum Movement

The optimum movement is a pre-recorded set of time series motion data of a person performing the same technique. It can be used for generating comparison data to the current performance. The optimum movement facilitates comparing metrics, generating a score, and comparing visualizations of the techniques.

The optimum movement may be a previous performance by the same user, a previous performance by another user, a combination of previous performances performed by the current user, a combination of performances by another user, or a combination of prior performances by many users. Alternatively, the optimum movement may be a manipulated version of a previous performance. For example, a coach may select a performance to become the optimum but alter the ankle, knee, and hip angles in order to adjust them to his or her liking. Another alternative may be that the optimum movement is computer generated. For example, the movement could be simulated on a computer and optimized in such a way that the optimum movement is found for that specific user.

Optimum Metrics

Optimum metrics are metrics generated from the optimum movement. Also, as in other areas, the metrics that are selected for use for the user may depend on and be customized according to the user profile and biometrics.

Scores

As the system allows detailed time series data in the sparse data streams to generate metrics, and also optimum movements to be developed which generate optimum metrics, the current and the optimum metrics may be compared. One result of comparing the metrics may be a score. The score may be a direct comparison of a metric from the current and the optimum metrics, or a combination of many metrics from the current and the optimum metrics. Further, the score may be a direct comparison of the values of the metrics or a function thereof. For example, where the metric being compared is the lead elbow flex at a given phase of the technique, the score may be calculated from the current metric value subtracted from the optimum metric value. It could also be normalized such as a percentage of the optimum metric.

The score could also be based on threshold difference values. For example, for the lead elbow flex in a given phase, when the current metric is within two degrees of the optimum metric, the system might identify that as “Great,” within five degrees as “Good,” ten degrees as “OK,” and 15 degrees as “Poor.” Further, the rating may be displayed to the user in a wide variety of methods such as color (e.g. green for great, yellow for good, orange for OK, and red for poor) or via a sound relating to the result (e.g. a verbal confirmation or equivalent noise). The threshold values may also be customized according to the user profile and the user biometrics. For example, an elite golfer may have very small threshold values, but a novice golfer may have larger ones. It may also be possible for the Coach/Expert or Users to set these thresholds according to preference (e.g. given the metric, phase of movement, user biometrics etc.).

Prioritized Feedback

The importance of the measures above can also be set by using summation filters that apply relative weights to a set of the scores and sum the weighted scores. Viewing this large data set through various summation filters (e.g. body segments, phases of motion, types of motion, evolution of performance over many repetitions) allows decisions to be made to present various audio-visual media as feedback to a user. This effectively “digitizes” a personal performance of a dynamic body motion skill. The hierarchical summarization of the information can be easily understood by the performer or a coach and used to suggest corrective action to achieve optimal performances in the future. The hierarchical structure of the output data, the tolerances/thresholds, and the weights can be customized using interchangeable configuration data files allowing different coaches (within a single sport/activity and for alternative sports/activities) to set their own importance and tolerances on body motion measurements.

Each individual metric is input to a function that yields a perfect score if it is between an inner pair of tolerance values and a zero score if it is outside an outer pair of tolerance values, and intermediate values yielding intermediate scores. The score can be signed (positive or negative) to indicate whether non-optimal performance was above or below the target value.

The calculated scores are then multiplied by their relative weights and summed into group summary scores according to the initial input configuration file. The group summary scores are themselves multiplied by group weights and summed to produce scores at a higher level in the hierarchy. The hierarchy need not be exactly tree-like with multiple different score summaries that re-use scores from a lower level. An example of this would be groupings by body category at a particular phase of the motion and groupings of phases for a single measurement.

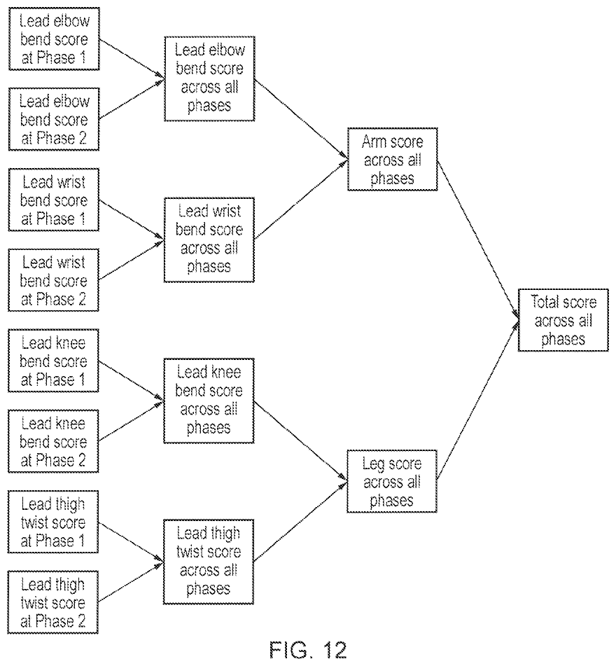

FIG. 12 is a block diagram illustrating one example of a hierarchical data structure of scores in some examples of the present disclosure. In the first (lowest) level, scores for different metric types are determined for each phase. For example, the following scores are determined: lead elbow bend score at phase 1, lead elbow bend know score at phase 2, lead wrist bend score at phase 1, lead wrist bend score at phase 2, lead knee bend score at phase 1, lead knee bend score at phase 2, lead thigh twist score at phase 1, and lead thigh twist score at phase 2. At the second level, summary scores for the same metric type across all phases are determined by weighting and summing the scores from prior level. For example, the following summary scores are determined: lead elbow bend scores across phases 1 and 2, lead wrist bend scores across phases 1 and 2, lead knee bend scores across phases 1 and 2, and lead thigh twist scores across phases 1 and 2. At the third level, summary scores of the same limb segment across all phases are determined by weighting and summing the scores from the prior level. For example, the following summary scores are determined: arm scores across phases 1 and 2 and leg scores across phases 1 and 2. At the fourth level, the total score across all phases are determined by weighting and summing the scores from the prior level.

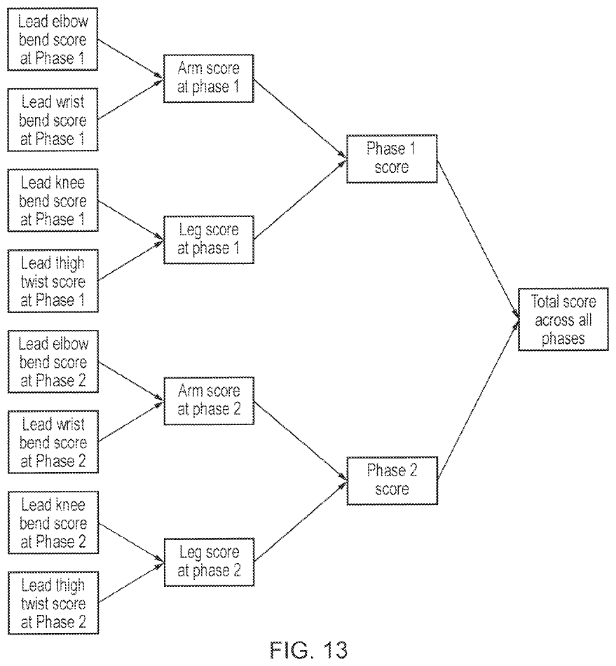

FIG. 13 is a block diagram illustrating another example of a hierarchical data structure of scores in some examples of the present disclosure. In the first (lowest) level, scores for different metric types are determined for each phase. For example, the following scores are determined: lead elbow bend score at phase 1, lead elbow bend know score at phase 2, lead wrist bend score at phase 1, lead wrist bend score at phase 2, lead knee bend score at phase 1, lead knee bend score at phase 2, lead thigh twist score at phase 1, and lead thigh twist score at phase 2. At the second level, summary scores for the same limb segment type in the same phase are determined by weighting and summing the scores from prior level. For example, the following summary scores are determined: arm scores at phase 1, leg scores at phase 1, arm score at phase 2, and leg score at phase 2. At the third level, summary scores of the phases are determined by weighting and summing the scores from the prior level. For example, the following summary scores are determined: phase 1 score and phase 2 score. At the fourth level, the total score across all phases are determined by weighting and summing the scores from the prior level.

A specific feedback hierarchy can be selected by determining the relative information content pertinent for the specific user. For example, a user that has a fault within his or her arm technique across all phases will exhibit a greater variability in his or her penultimate score layer (3rd column) for the hierarchy in FIG. 12 compared to the variability in the penultimate score layer for the hierarchy in FIG. 13, which will be relatively small. Therefore, the hierarchy in FIG. 12 is more informative for that user and should be selected. Alternatively, a second user may be balanced in their scoring for arms and legs but have variability across the duration of a swing: performing poorly, in Phase 1, but performing well in Phase 2. For this second user, the hierarchy in FIG. 13 is more informative and should be selected for visual feedback.

The final output is a hierarchical self-describing data structure may be in JavaScript Object Notation (JSON) format that can be transmitted to a separate device (from that on which the calculations are carried out) for display on a suitable Graphical User Interface (GUI)

Visualized Feedback

Once the phase recognition had been passed, it is assumed that the current data window contains data for the entire technique of interest.

In order to transmit the window of data representing a mathematical model of movement over time across a low bandwidth connection to app 134 on a smart device, application controller 130 compresses the data by exploiting the features of motion capture data to achieve a large reduction in data size. To minimize the amount of processing required for the geometric data used in the previously described activity executables 132 (FIG. 1), the quaternions of the limb segments are expressed in a coordinate reference system that is common to all the segments. However, the motions of the joined segments of a skeletal model are related to one another. Therefore, application controller 130 converts the quaternion describing a segment orientation from a common reference coordinate system to a coordinate system relative to the orientation of an attached or “parent” limb segment because the rate of change of the relative quaternion is significantly lower than that of the common reference quaternion. Therefore, the application of the same compression criteria as for the original quaternions (sufficient change in orientation from a previous value, or sufficient time gap) results in a greater compression ratio. The accuracy of the decompressed data for generating the avatar can be specified according to time gap duration or change in relative orientation of the segment.

As an example, during the majority of a golf swing, the motion of both forearm segments is similar to their connected upper arm segments. If one considers a camera mounted to the upper arm, looking at the forearm during the swing, the view of the forearm motion will be slower than the background of the image, which would be moving fast. Therefore, by describing the motion of the forearm relative to the upper arm rather than relative to the environment (absolute to a common coordinate reference system), a smaller number of quaternions (orientations), spaced further apart in time can be used to accurately describe the motion of the forearm when the motion of the upper arm is taken into account. In other words, because the movement of a child segment relative to its parent segment is likely to be less than its overall movement in space, the method used to create the spare geometric data uses this to save space (stored data) as only changes greater than certain thresholds 9 (e.g., 1 degree per second or 1 second) are saved.

This relative quaternion representation also has the advantage of requiring less processing when used to drive a visualization of the data as an animation because it more closely matches the representations used in common 3D graphics languages such as OpenGL. The quaternions (containing 4 numbers represented in 8 bytes for a total of 32 bytes are also converted to rotation vectors requiring only three (3) components. The three (3) vector components are then scaled and converted to signed two (2) byte integers for a total of only 6 bytes per orientation.

The time series data from both the current data and the optimum data may be aligned (temporarily and/or spatially) and reconstructed in order to make a visual human (avatar) representation of the action. These may then be compared in order for the user to see differences between the data. The score data may also be used to enhance this comparison such as through the addition of angle or distance notation to highlight particular areas of interest.

Multiple systems may be synchronized in order to track the movements of the whole or part of the bodies of multiple users synchronously such as when users may be performing at the same time, against each other (e.g. game of tennis or pitching and batting in baseball). This permits analysis of individuals within the team, sub-sections of a team, and the whole team overall. For example, in rowing, the performance of an individual rower may be analyzed in terms of their rowing technique but also in terms of their contribution to the performance of the team overall (e.g., timing of technique, power output etc.). Further, this analysis may be provided (and compared) for the rowers on the port and/or starboard sides. This analysis can also drive feedback to the individuals, group or team accordingly. For example, in the rowing case, the system may analyze that the timing of the group is not optimal and hence provides audio feedback to the whole group.

In the example above, it is not important to know the relative location of each performer/user, other than his or her position in the boat. In other situations, the addition of location data will assist the analysis of the movements and relative movements of members of the same team, or people competing on opposite teams. This is because whole body movements or partial body movements, may be analyzed in conjunction with location data. Where the multiple users are opponents who are both using the system, it becomes possible to see the positions, movements, and timings of how each user reacts to their opponent. For example, in tennis it would be possible to see how and when one user served and then how and when the other player responds.

In order to gain the location data, GPS is commonly used for such a purpose. As sensors 114 are interchangeable, one of the sensors, e.g. the upper spine, may be swapped out for one that includes a GPS device. Alternatively, the GPS unit may be included into the pod 118 or gained from the smart device with app 134 in instances where this might be carried during the activity (e.g. golf).

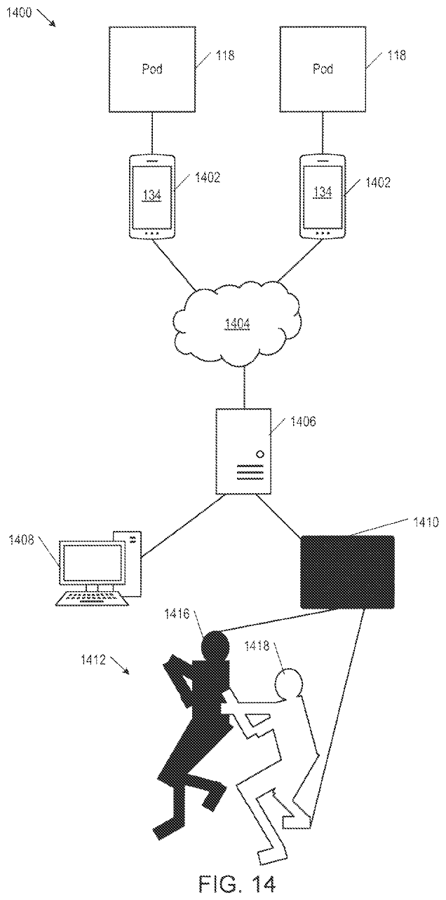

FIG. 14 is a block diagram illustrating an application 1400 of motion system 100 (FIG. 1) to analyze motion data from multiple users in some examples of the present disclosure. Two pods 118 capture motion of two users and transmit sparse geometric data streams to their respective apps 134 on phones 1402, which upload the sparse geometric data over the Internet 1404 to a server computer 1406 of provider 202 (FIG. 1). Alternatively, pods 118 directly upload the sparse geometric data to server computer 1406. The users may be two crew members rowing or two basketball players playing one-on-one. Server computer 1406 has the necessary process and memory to process the sparse geometric data to create avatars of the two users. Specifically, server computer 1406 generates a video 1412 by temporarily or spatially aligning the two avatars 1414 and 1416 and transmitting video 1412 to a computer 1408 or a tablet 1410 for a coach to view and provide feedback to the users. Alternatively, server computer 1406 may automatically generate feedback from scoring the sparse geometric data of the users. For example, server computer 1406 may determine synchronization scores in the phases of a rowing motion by determining and comparing metrics from the sparse geometric data of the users. Server computer 1406 may then provide feedback identifying phases that lack synchronization. For example, sever computer 1406 may transmit video 1412 of avatars 1414 and 1416 along with identification of the phases lacking synchronization, which may appear in the form of text or audio in video 1412, to phones 1402 of the users.

Various other adaptations and combinations of features of the examples disclosed are within the scope of the invention. Numerous examples are encompassed by the following claims.

What is claimed is:

1: A method for a processor to detect and analyze an action, comprising:
receiving or retrieving information about a user, hereafter the user information;
receiving times series of raw motion data, hereafter the raw data streams, generated by corresponding sensors on the user;
generating sparse data streams based on the raw data streams;
performing a raw data action identification to detect the action in a window of time, comprising:
modifying one or more first thresholds based on the user information; and
comparing one or more of the raw data streams in the window with one or more modified first thresholds to detect the action; and
when the action is detected, providing a visual feedback to the user based on the sparse data streams.

2: The method of claim 1, where the user information comprises:
user profile including one or more of gender, date of birth, ethnicity, location, skill level, recent performance data, and health information; or
user biometrics including one or more of passive range of movement at each joint, active range of movement at each joint, strength indicator, anthropometric measurements, resting heart rate, and breathing rates.

3: The method of claim 1, further comprising receiving a selection of an action from the user.

4: The method of claim 1, wherein said receiving times series of raw motion data further comprising receiving a raw data stream generated by a corresponding sensor on a piece of equipment used by the user in performing the action.

5: The method of claim 1, wherein generating the sparse data streams comprises:
determining time series of sensor orientation, hereafter the sensor orientation streams, for the corresponding sensors from the raw data streams;
determining time series of segment orientation, hereafter the segment orientation streams, for corresponding limb segments of a skeletal model from the sensor orientations streams; and
determining the sparse data streams from the corresponding segment orientation streams.

6: The method of claim 5, wherein determining the sparse data streams from the corresponding segment orientation streams comprises, for each sparse data stream:
from each segment orientation in the corresponding segment orientation stream, determining if (1) the segment orientation changed by more than an orientation threshold from a prior segment orientation in the corresponding segment orientation stream or (2) the segment orientation occurred more than a time threshold after the prior segment orientation in the corresponding segment orientation stream, wherein the segment threshold and the time threshold are specific to the action; and
when condition (1) or (2) is met, adding the segment orientation to the sparse data stream.

7: The method of claim 6, wherein each segment orientation in the sparse data streams comprises a quaternion that describe an orientation of a limb segment relative to another limb segment.

8: The method of claim 1, further comprising:
when the raw data action identification detects the action in the window, performing a geometric data action identification to determine the action in the window; and
when the geometric data action identification detects the action in the window, detecting phases of the action.

9: The method of claim 8, wherein said performing a geometric data action identification comprises:
modifying one or more second thresholds and one or more third thresholds based on the user information; and
(1) comparing one or more of the raw data streams in the window with one or more second thresholds and (2) comparing one or more of the sparse data streams in the window with one or more third thresholds.

10: The method of claim 7, wherein said detecting phases of the action comprises:
modifying one or more fourth thresholds and one or more fifth thresholds based on the user information; and
(1) comparing one or more of the raw data streams in the window with one or more third fourth thresholds and (2) comparing one or more of the sparse data streams in the window with one or more fourth thresholds.

11: The method of claim 7, further comprising:
when a number of the phases of the action is not detected, selecting the next window in time to detect the action;
when the number of the phases is detected:
determining current metrics in the phases from one or more of the sparse data streams; and
determining a score of the detected action, comprising:
modifying optimum metrics based on the user information; and
comparing the current metrics against the optimum metrics.

12: The method of claim 11, further comprising selecting the current metrics from all available metrics based on the user information.

13: The method of claim 11, wherein the optimum metrics are based an optimum performance of the action, the optimum performance is one or a combination of prior performances by the user, another user, or a combination of users, or the optimum performance is selected from a set of optimum performances edited by coaches.

14: The method of claim 11, further comprising prioritizing feedbacks to the user by:
determining multiple scores for the action in the time window, comprising, for each of the current metrics, providing a first score when the current metric is within a first range of a corresponding optimum metric, a second score when the current metric is within a second range of the corresponding optimum metric, and a third score when the current metric is outside of the second range, the first score being signed to indicate if the corresponding current metric is greater or less than the optimum metric;
multiplying the scores with corresponding weights;
summing groups of the weighted scores to generate group summary scores;
multiplying the group summary scores by corresponding weights; and
summing supergroups of the weighted group summary scores to generate supergroup summary scores.

15: The method of claim 11, wherein said providing visualized feedback to the user, comprises creating a visual comparison between the action in the window and an optimum performance based on the optimum metrics, comprising:
creating a first visual representation from the sparse data streams;
creating a second visual representation from the optimum performance; and
temporarily or spatially aligning the first and the second visual representations.

16: The method of claim 15, further comprising enhancing the visual comparison by indicating angle or distance notations based on the scores to highlight areas of interest.

17: The method of claim 1, wherein the action comprises a skill, a technique of a skill, a variation of a technique, or a pose.

18: A method for a processor to analyze actions from multiple users, comprising:
receiving at least a first plurality of sparse data streams from a first user and a second plurality of spare data streams from a second user, each sparse data stream comprising at least a portion of motion data recorded at irregular intervals;
creating a first visual representation of the first user from the first plurality of sparse data streams;
creating a second visual representation of the second user from the second plurality of sparse data streams;
temporarily or spatially aligning the first and the second visual representations; and
generating a video comprising the temporarily or spatially aligned first and the second visual representations.

19: The method of claim 18, further comprising determining a score based on movement synchronization based on the first and the second pluralities of sparse data streams.

20: The method of claim 19, further comprising transmitting the video and a feedback to at least one of the first and the second users based on the score.
